(19) United States
(12) Patent Application Publication
(10) Pub. No.: US 2024/0197651 A1
(43) Pub. Date: Jun. 20, 2024

(54) DEXMECAMYLAMINE AND USES THEREOF

(71) Applicant: Atacama Therapeutics, Inc., Wellesley, MA (US)

(72) Inventors: Joseph A. BARON, Wellesley, MA (US); Jonathan REIS, Brookline, MA (US)

(21) Appl. No.: 18/572,977

(22) PCT Filed: Jun. 24, 2022
(86) PCT No.: PCT/US2022/034932

§ 371 Date: Dec. 21, 2023

Related U.S. Application Data
(60) Provisional application No. 63/214,567, filed on Jun. 24, 2021.

(51) Int. Cl. A61K 31/13; A61P 17/08; A61P 17/10; A61P 35/00

(52) U.S. Cl. CPC A61K 31/13; A61P 17/08; A61P 17/10; A61P 35/00

(57) ABSTRACT

The disclosure features methods of treating a subject with acne, enlarged pores, hidradenitis, seborrhea, sebaceous hyperplasia, seborrheic dermatitis, sebaceous adenoma, sebaceous carcinoma, sebaceous cyst, wrinkles, or oily skin suppurativa by administration of a dexmecamylamine substantially free of exo-R-mecamylamine, or a pharmaceutically acceptable salt thereof to the subject.

### SUMMARY OF THE DISCLOSURE

The present disclosure provides methods e.g., administration of a compound (e.g., dexmecamylamine substantially free of exo-R-mecamylamine), for treating disorders in patients.

In one aspect, the disclosure features a method of treating acne, enlarged pores, hidradenitis suppurativa, seborrhea, sebaceous hyperplasia, seborrheic dermatitis, sebaceous adenoma, sebaceous carcinoma, sebaceous cyst, wrinkles, or oily skin (e.g., acne, hidradenitis suppurativa, seborrhea, sebaceous hyperplasia, seborrheic dermatitis, sebaceous adenoma, sebaceous carcinoma, sebaceous cyst, wrinkles, or oily skin, or acne or hidradenitis suppurativa) in a subject in need thereof. The method includes administering a therapeutically effective amount of dexmecamylamine substantially free of exo-R-mecamylamine, or a pharmaceutically acceptable salt thereof, to the subject.

In some embodiments, the method includes administering a therapeutically effective amount of dexmecamylamine substantially free of exo-R-mecamylamine, or a pharmaceutically acceptable salt thereof, to a subject with acne (e.g., without enlarged pores). In some embodiments, the method includes administering a therapeutically effective amount of dexmecamylamine substantially free of exo-R-mecamylamine, or a pharmaceutically acceptable salt thereof, to a subject with enlarged pores. In some embodiments, the method includes administering a therapeutically effective amount of dexmecamylamine substantially free of exo-R-mecamylamine, or a pharmaceutically acceptable salt thereof, to a subject with hidradenitis suppurativa. In some embodiments, the method includes administering a therapeutically effective amount of dexmecamylamine substantially free of exo-R-mecamylamine, or a pharmaceutically acceptable salt thereof, to a subject with seborrhea. In some embodiments, the method includes administering a therapeutically effective amount of dexmecamylamine substantially free of exo-R-mecamylamine, or a pharmaceutically acceptable salt thereof, to a subject with sebaceous hyperplasia. In some embodiments, the method includes administering a therapeutically effective amount of dexmecamylamine substantially free of exo-R-mecamylamine, or a pharmaceutically acceptable salt thereof, to a subject with seborrheic dermatitis. In some embodiments, the method includes administering a therapeutically effective amount of dexmecamylamine substantially free of exo-R-mecamylamine, or a pharmaceutically acceptable salt thereof, to a subject with sebaceous adenoma. In some embodiments, the method includes administering a therapeutically effective amount of dexmecamylamine substantially free of exo-R-mecamylamine, or a pharmaceutically acceptable salt thereof, to a subject with sebaceous carcinoma. In some embodiments, the method includes administering a therapeutically effective amount of dexmecamylamine substantially free of exo-R-mecamylamine, or a pharmaceutically acceptable salt thereof, to a subject with sebaceous cyst. In some embodiments, the method includes administering a therapeutically effective amount of dexmecamylamine substantially free of exo-R-mecamylamine, or a pharmaceutically acceptable salt thereof, to a subject with wrinkles. In some embodiments, the method includes administering a therapeutically effective amount of dexmecamylamine substantially free of exo-R-mecamylamine, or a pharmaceutically acceptable salt thereof, to a subject with oily skin.

In some embodiments, the dexmecamylamine substantially free of exo-R-mecamylamine, or pharmaceutically acceptable salt thereof, is administered by way of oral, inhalational, intranasal, or topical administration. In some embodiments, if the subject has enlarged pores, the dexmecamylamine substantially free of exo-R-mecamylamine is administered orally.

In some embodiments, the subject is administered 0.1 mg to 16 mg (e.g., 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, or 16 mg) of the dexmecamylamine substantially free of exo-R-mecamylamine, or pharmaceutically acceptable salt thereof.

In some embodiments, the dexmecamylamine substantially free of exo-R-mecamylamine, or pharmaceutically acceptable salt thereof is administered at a dose of about 1, 2, 4, or 8 mg to the subject. In some embodiments, the subject is administered 2 mg of the dexmecamylamine substantially free of exo-R-mecamylamine, or pharmaceutically acceptable salt thereof. In some embodiments, the subject is administered 4 mg of the dexmecamylamine substantially free of exo-R-mecamylamine, or pharmaceutically acceptable salt thereof.

In some embodiments, the dexmecamylamine substantially free of exo-R-mecamylamine is a salt form, e.g., the hydrochloride salt.

In some embodiments, the subject produces sebum in an amount of 100 μg/cm² or greater (e.g., 125, 135, 150, 175, 200 μg/cm² or higher). In some embodiments, following the treatment with dexmecamylamine substantially free of exo-R-mecamylamine, or pharmaceutically acceptable salt thereof, the subject exhibits sebum production that is at least 20% (e.g., at least 20%, 25%, 30%, 35%, 40%, 45%, 50%, 55%, 60%, 65%, 70%, 75%, 80%, 85%, 90%, 95%, 96%, 97%, 98%, 99% or more) less than the sebum production in the subject prior to the treatment with the dexmecamylamine substantially free of exo-R-mecamylamine, or pharmaceutically acceptable salt thereof. In some embodiments, the sebum production is facial sebum production.

In some embodiments, the subject is a human.

In some embodiments, treatment is for at least one week, e.g., at least two, three, or four weeks or at least 2, 3, 6, or 12 months.

### Definitions

As used herein, the terms “about” and “approximately” refer to a value that is within 10% above or below the value being described. For example, the term “about 5 nM” indicates a range of from 4.5 to 5.5 nM.

As used herein, the term “administration” refers to the administration of a composition (e.g., a compound or a preparation that includes a compound as described herein) to a subject or system. Administration to an animal subject (e.g., to a human) may be by any appropriate route. For example, in some embodiments, administration may be bronchial (including by bronchial instillation), inhalational buccal, enteral, interdermal, intra-arterial, intradermal, intragastric, intramedullary, intramuscular, intranasal, intraperitoneal, intrathecal, intratumoral, intravenous, intraventricular, mucosal, nasal, oral, rectal, subcutaneous, sublingual, topical, tracheal (including by intratracheal instillation), transdermal, vaginal, or vitreal.

For reference herein, “dexmecamylamine substantially free of exo-R-mecamylamine” includes dexmecamylamine in amounts greater than or equal to 95% by weight and exo-R-mecamylamine 5% or less by weight. More preferably, “dexmecamylamine substantially free of exo-R-mecamylamine” includes dexmecamylamine greater than or equal to 98% by weight and exo-R-mecamylamine 2% or less by weight. More preferably, “dexmecamylamine substantially free of exo-R-mecamylamine” includes dexmecamylamine greater than or equal to 99% by weight and exo-R-mecamylamine 1% or less by weight. Even more preferably, “dexmecamylamine substantially free of exo-R-mecamylamine” includes dexmecamylamine greater than or equal to 99.5% by weight and exo-R-mecamylamine 0.5% or less by weight. Most preferably, “dexmecamylamine substantially free of exo-R-mecamylamine” includes dexmecamylamine greater than or equal to 99.7% by weight and exo-R-mecamylamine 0.3% or less by weight.

The term “pharmaceutical composition,” as used herein, represents a composition containing a compound described herein formulated with a pharmaceutically acceptable excipient. Pharmaceutical compositions can be formulated, for example, for oral administration in unit dosage form (e.g., a tablet, capsule, caplet, gelcap, transbuccal, or syrup); for topical administration (e.g., as a cream, gel, lotion, dermal patch, or ointment); for intravenous administration (e.g., as a sterile solution free of particulate emboli and in a solvent system suitable for intravenous use); or in any other pharmaceutically acceptable formulation.

A “pharmaceutically acceptable excipient,” as used herein, refers to any ingredient other than the compounds described herein (for example, a vehicle capable of suspending or dissolving the active compound) and having the properties of being substantially nontoxic and non-inflammatory in a patient. Excipients include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensions, or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

As used herein, the term “pharmaceutically acceptable salt” means any pharmaceutically acceptable salt of the compounds described herein. For example, pharmaceutically acceptable salts of any of the compounds described herein include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting a free base group with a suitable organic acid.

The compounds of the disclosure may have ionizable groups so as to be capable of preparation as pharmaceutically acceptable salts. These salts may be acid addition salts involving inorganic or organic acids. Frequently, the compounds are prepared or used as pharmaceutically acceptable salts prepared as addition products of pharmaceutically acceptable acids. Suitable pharmaceutically acceptable acids and methods for preparation of the appropriate salts are well-known in the art. Salts may be prepared from pharmaceutically acceptable non-toxic acids and including inorganic and organic acids.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate salts.

As used herein, the term “subject” refers to any organism to which a composition in accordance with the disclosure may be administered, e.g., for experimental, diagnostic, prophylactic, and/or therapeutic purposes. Typical subjects include any animal (e.g., mammals such as mice, rats, rabbits, non-human primates, and humans). A subject may seek or be in need of treatment, require treatment, be receiving treatment, be receiving treatment in the future, or be a human or animal who is under care by a trained professional for a particular disease or condition.

As used herein, the terms “treat,” “treated,” or “treating” mean both therapeutic treatment and prophylactic or preventative measures wherein the object is to prevent or slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment.

The details of one or more embodiments of the disclosure are set forth in the description below. Other features, objects, and advantages of the disclosure will be apparent from the description and from the claims.

### DETAILED DESCRIPTION OF THE DISCLOSURE

The disclosure provides methods of treating various diseases or disorders (e.g., acne, enlarged pores, hidradenitis suppurativa, seborrhea, sebaceous hyperplasia, seborrheic dermatitis, sebaceous adenoma, sebaceous carcinoma, sebaceous cyst, wrinkles, and oily skin) in a patient with dexmecamylamine substantially free of exo-R-mecamylamine, or a pharmaceutically acceptable salt thereof. A subject having acne, enlarged pores, hidradenitis suppurativa seborrhea, sebaceous hyperplasia, seborrheic dermatitis, sebaceous adenoma, sebaceous carcinoma, sebaceous cyst, wrinkles, or oily skin may be treated according to the methods of the disclosure by administering to the subject an effective amount of dexmecamylamine substantially free of exo-R-mecamylamine, or a pharmaceutically acceptable salt thereof by any route described herein.

### Pharmaceutical Compositions

The compounds of the disclosure are preferably formulated into pharmaceutical compositions for administration to human subjects in a biologically compatible form suitable for administration in vivo.

The compound of the disclosure may be used in the form of the free base, or in the form of salts. All forms are within the scope of the disclosure. In accordance with the methods of the disclosure, the described compound, or pharmaceutically acceptable salts thereof, may be administered to a patient in a variety of forms depending on the selected route of administration, as will be understood by those skilled in the art. The compound of the disclosure may be administered, for example, by oral, parenteral, buccal, topical, sublingual, nasal, inhalational (e.g., by way of a nebulizer), rectal, patch, pump, or transdermal administration and the pharmaceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time.

The present disclosure includes a salt or solvate of the compound herein described, including combinations thereof such as a solvate of a salt. The compound may exist in solvated, for example hydrated, as well as unsolvated forms, and the present disclosure encompasses all such forms. Typically, but not absolutely, the salts of the present disclosure are pharmaceutically acceptable salts. Salts encompassed within the term “pharmaceutically acceptable salts” refer to non-toxic salts of the compounds of this disclosure. Examples of suitable pharmaceutically acceptable salts include inorganic acid addition salts such as chloride, bromide, sulfate, phosphate, and nitrate; organic acid addition salts such as acetate, galactarate, propionate, succinate, lactate, glycolate, malate, tartrate, citrate, maleate, fumarate, methanesulfonate, p-toluenesulfonate, and ascorbate; and salts with acidic amino acid such as aspartate and glutamate. The salts may be in some cases hydrates or ethanol solvates. In certain embodiments, dexmecamylamine hydrochloride is a preferential salt form.

Although it is possible to administer the compound of the present disclosure in the form of a bulk active chemical, it is preferred to administer the compound in the form of a pharmaceutical composition or formulation. Thus, one aspect of the present disclosure includes pharmaceutical compositions comprising the compound (e.g., dexmecamylamine substantially free of exo-R-mecamylamine) and/or pharmaceutically acceptable salts thereof and one or more pharmaceutically acceptable carriers, diluents, or excipients. Another aspect of the disclosure provides a process for the preparation of a pharmaceutical composition including admixing the compound of the disclosure (e.g., dexmecamylamine substantially free of exo-R-mecamylamine) and/or pharmaceutically acceptable salts thereof with one or more pharmaceutically acceptable carriers, diluents or excipients.

A compound of the disclosure may be orally administered, for example, with an inert diluent or with an assimilable edible carrier, or it may be enclosed in hard- or soft-shell gelatin capsules, or it may be compressed into tablets, or it may be incorporated directly with the food of the diet. For oral therapeutic administration, a compound of the disclosure may be incorporated with an excipient and used in the form of oral, buccal or sublingual thin film, ingestible tablets, buccal tablets, troches, capsules, elixirs, suspensions, syrups, and wafers.

A compound of the disclosure may also be administered parenterally. Solutions of a compound of the disclosure can be prepared in water suitably mixed with a surfactant, such as hydroxypropylcellulose. Dispersions can also be prepared in glycerol, liquid polyethylene glycols, DMSO and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington: The Science and Practice of Pharmacy (2020, 23^{rd} ed.) and in The United States Pharmacopeia: The National Formulary (USP43-NF38). For example, the dexmecamylamine substantially free of exo-R-mecamylamine can be administered topically. Topical formulations of the compound can include between 0.05% to 1% (w/w) of the compound, e.g., about 0.1% (w/w) of the compound.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that may be easily administered via syringe.

Compositions for nasal administration may conveniently be formulated as aerosols, drops, gels, and powders. Aerosol formulations typically include a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device, such as a single dose nasal inhaler or an aerosol dispenser fitted with a metering valve which is intended for disposal after use. Where the dosage form comprises an aerosol dispenser, it will contain a propellant, which can be a compressed gas, such as compressed air or an organic propellant, such as fluorochlorohydrocarbon. The aerosol dosage forms can also take the form of a pump-atomizer.

Pharmaceutical compositions may be formulated in unit dose form, or in multiple or subunit doses. The pharmaceutical compositions may be administered to a patient or subject such as a mammal such as a mouse, rat, cat, rabbit, dog, pig, cow, or monkey; but advantageously is administered to a human being. In addition, the time of day (e.g., morning, evening, before bedtime) and the number of times per day (e.g., once per day, twice per day) that the pharmaceutical composition is administered can vary.

In some embodiments, the unit dosage is about 1-8 mg, e.g., about 1 mg, 2 mg, 3 mg, 4 mg, 5 mg, 6 mg, 7 mg, or 8 mg. For example, an oral dosage form may include 2 mg, 4 mg, or 8 mg of dexmecamylamine substantially free of exo-R-mecamylamine.

### Therapeutic Methods

The inventors have discovered that dexmecamylamine reduces sebum production, which indicates the dexmecamylamine can be used in the treatment of diseases and disorders caused or exacerbated by oily skin. Such conditions include acne, enlarged pores, hidradenitis suppurativa, seborrhea, sebaceous hyperplasia, seborrheic dermatitis, sebaceous adenoma, sebaceous carcinoma, sebaceous cyst, wrinkles, and oily skin.

For example, the compound of the disclosure (e.g., dexmecamylamine substantially free of exo-R-mecamylamine, or a pharmaceutically acceptable salt thereof) is effective in treating acne. Acne, also known as acne vulgaris, is a chronic disorder affecting the hair and follicle sebaceous gland, where there is expansion and blockage of the follicle and resultant inflammation. Acne is characterized by blackheads or whiteheads, pimples, oily skin, and possible scarring. There are several variants of acne, such as comedonal acne and nodulocystic acne. Enlarged pores can be associated with acne, as well as other conditions such as rosacea, and is typically associated with sebum production, skin aging and photodamage, and hair follicle size.

The compound of the disclosure (e.g., dexmecamylamine substantially free of exo-R-mecamylamine, or a pharmaceutically acceptable salt thereof,) can also be administered to a subject to treat a symptom of any one of the diseases or disorders described herein. In some embodiments, the subject does not have hyperhidrosis, overactive bladder (OAB), substance addiction (e.g., to nicotine, cocaine, alcohol, amphetamine, opiate, other psychostimulant and a combination thereof), hypertension, hypertensive crisis, herpes type I and II, Tourette's Syndrome or other tremors, cancer (such as small cell lung cancer), atherogenic profile, neuropsychiatric disorders (such as bipolar disorder, depression, anxiety disorder, panic disorder, schizophrenia, seizure disorders, Parkinson's disease and attention deficit hyperactivity disorder), chronic fatigue syndrome, Crohn's disease, autonomic dysreflexia, or spasmogenic intestinal disorders and/or does not smoke.

To treat any of the conditions described herein, the dexmecamylamine substantially free of exo-R-mecamylamine, or a pharmaceutically acceptable salt thereof, can be administered to the patient once daily. Alternatively, the dexmecamylamine substantially free of exo-R-mecamylamine, or a pharmaceutically acceptable salt thereof, can be administered two, three, four, five, or more times daily, as necessary for effective treatment of the condition.

Treatment may occur for as long as necessary to reach or maintain a desired therapeutic outcome. For example, treatment may occur for at least one week, such as at least two, three or four weeks, or two, three, four, five, or six months, or one year or more. Treatment may occur once or more daily, e.g., twice or three times.

In some embodiments, the subject is diagnosed with acne, enlarged pores, hidradenitis suppurativa, seborrhea, sebaceous hyperplasia, seborrheic dermatitis, sebaceous adenoma, sebaceous carcinoma, sebaceous cyst, wrinkles, or oily skin prior to treatment.

### Dosages

The dosage of the compound of the disclosure or compositions comprising the compound can vary depending on multiple factors, such as, e.g., the pharmacodynamic properties of the compound, the mode of administration, age, health, or weight of the recipient, the nature and extent of the symptoms, frequency of the treatment, the type of concurrent treatment, if any, and the clearance rate of the compound in the animal to be treated. One of skill in the art can determine the appropriate dosage based on the above factors. The compound of the disclosure may be administered initially in a suitable dosage that may be adjusted as required, depending on the clinical response. In general, satisfactory results may be obtained when the compounds of the disclosure are administered to a human at a daily dosage of, for example, between 0.05 mg and 3000 mg (measured as the solid form). Dose ranges include, for example, between 0.1-1000 mg (e.g., 0.2-950, 0.4-900, 0.6-850, 0.8-800, 1-750, 1-20, 2-16, 2-700, 4-650, 6-600, 8-550, 10-500, 15-450, 20-400, 30-350, 40-300, 50-250, 75-200, or 100-150 mg). In some embodiments, about 1, 2, 2.5, 4, 5, 8, 10, 15, or 20 mg of the dexmecamylamine substantially free of exo-R-mecamylamine, or a pharmaceutically acceptable salt thereof is administered, e.g., per day. A single dose or multiple doses may be administered in a 24-hour period. For example, in certain embodiments, between 0.5-8 mg (e.g., 0.5, 1, 1.5, 2, 2.5, 3, 3.5, 4, 4.5, 5, 5.5, 6, 6.5, 7, 7.5, or 8 mg) of the compound is administered to the subject once or more than once, e.g., twice, daily.

Alternatively, the dosage amount can be calculated using the body weight of the patient. For example, the dose of a compound, or pharmaceutical composition thereof, administered to a patient may be 0.005-5 (0.01-4.8, 0.02-4.6, 0.04-4.4, 0.06-4.2, 0.08-4.0, 0.1-3.8, 0.2-3.6, 0.3-3.4, 0.4-3.2, 0.5-3.0, 0.6, 2.8, 0.7-2.6, 0.8-2.4, 0.9-2.2, 1-2, 1.1-1.9, 1.2-1.8, 1.3-1.7, or 1.4-1.6) mg/kg. In exemplary, non-limiting embodiments, the dose may range from 0.005-1 mg/kg (e.g., 0.01-0.5, 0.01-0.2, or 0.01-0.1 mg/kg).

### EXAMPLES

The following examples are put forth so as to provide those of ordinary skill in the art with a description of how the compositions and methods described herein may be used, made, and evaluated, and are intended to be purely exemplary of the disclosure and are not intended to limit the scope of what the inventors regard as their invention.

### Example 1. Phase II Clinical Study of Human Subjects Treated with Dexmecamylamine

Acne vulgaris is a dermatological condition characterized by increased sebum production, which is driven, in part, by cholinergic signaling within the sympathetic nervous system. Activation of nicotinic receptors on human sebaceous glands is known to increase sebum production and secretion. Dexmecamylamine acts on nicotinic acetylcholine receptors to reduce sympathetic tone through the selective inhibition of presynaptic nicotinic receptors in the sympathetic ganglia. In order to determine the therapeutic efficacy of dexmecamylamine in the treatment of acne and related conditions, the inventors leveraged a Phase II clinical study in 15 human subjects with hyperhidrosis and oily skin (i.e., sebum production of 100 μg/cm² or greater (e.g., 125, 135, 150, 175, 200 μg/cm² or higher)) that were treated with dexmecamylamine hydrochloride. Subjects treated with 2 mg or 4 mg BID of dexmecamylamine exhibited a reduction in facial sebum production following four weeks of treatment with dexmecamylamine, as is shown in Table 1, below.

TABLE 1
Sebum production in human subjects
treated with dexmecamylamine

| | | Average | | Average |
|---|---|---|---|---|
| Treatment | # | response category | Responders | response category |
| Group | Subjects | at 2 weeks* | (%)*** | at 4 weeks** |
| Placebo | 5 | Low^{†} | 40% | Low |
| 2 mg BID | 5 | Low | 80% | Medium^{††} |
| 4 mg BID | 5 | Low | 100% | High^{†††} |

*Response is measured as the change in percent of average facial sebum quantity at the end of a 2-week treatment regimen as compared to baseline sebum production.
**Response is defined as the change in percent of average facial sebum quantity at the end of a 4-week treatment regimen as compared to baseline sebum production.
***Responders are defined as subjects that exhibit 20% or greater reduction in sebum quantity following treatment with dexmecamylamine.
^{†}Reduction in sebum production of less than 20%.
^{††}Reduction in sebum production between 20% and 35%.
^{†††}Reduction in sebum production of greater than 35%.

These findings demonstrate that dexmecamylamine may be advantageously used as a therapeutic agent for the treatment of conditions associated with excessive sebum production, including, e.g., acne, enlarged pores, hidradenitis suppurativa, seborrhea, sebaceous hyperplasia, seborrheic dermatitis, sebaceous adenoma, sebaceous carcinoma, sebaceous cyst, wrinkles, or oily skin, especially with treatment of greater than two weeks.

### Example 2. Treatment of Acne

The compound of the disclosure (e.g., dexmecamylamine substantially free of exo-R-mecamylamine or pharmaceutically acceptable salt thereof) is administered to patients with acne. Patients receiving the compound exhibit improved symptoms, where signs of acne, including whiteheads, blackheads, papules, pimples, nodules, and cysts are reduced or eliminated as a result of administration of the compound. In some embodiments, the compound is dexmecamylamine substantially free of exo-R-mecamylamine, or a pharmaceutically acceptable salt thereof. In some embodiments, the subject produces sebum in an amount of 100 μg/cm² and above (e.g., 125, 135, 150, 175, 200 μg/cm² or higher).

In particular, 4 mg of dexmecamylamine hydrochloride is administered orally twice a day to a subject. Subjects have oily skin (>100 μg/cm²) as measured on three locations of the face by a Sebumeter® grease spot photometry device and has facial acne vulgaris (including the nose). Efficacy is determined by a comparison of sebum as an absolute change from baseline at 12 weeks. Other measures of efficacy include the absolute change from baseline in inflammatory and non-inflammatory lesion counts at 8 weeks and the percent change from baseline in lesion counts (inflammatory and noninflammatory) at weeks 8 and 12. A further measure of efficacy is qualitative assessment on a five point scale (0-4) as a change from baseline at 12 weeks; success may be defined as a grade of 0 or 1 with at least a two grade improvement. The assessment may include the quantity of lesions, size of lesions, intensity of inflammation, and location of lesions to consider the quality and quantity of lesions.

### Example 3. Treatment of Enlarged Pores

The compound of the disclosure (e.g., dexmecamylamine substantially free of exo-R-mecamylamine) is administered to patients with enlarged pores. Patients receiving the compound exhibit improved symptoms, where enlarged pores are reduced or eliminated as a result of administration of the compound. In some embodiments, the compound is dexmecamylamine substantially free of exo-R-mecamylamine, or a pharmaceutically acceptable salt thereof. In some embodiments, the subject produces sebum in an amount of 100 μg/cm² and above (e.g., 125, 135, 150, 175, 200 μg/cm² or higher).

### Example 4. Treatment of Hidradenitis Suppurativa

The compound of the disclosure (e.g., dexmecamylamine substantially free of exo-R-mecamylamine) is administered to patients with hidradenitis suppurativa. Patients receiving the compound exhibit improved symptoms, where hidradenitis suppurativa is reduced or ceased as a result of administration of the compound. In some embodiments, the subject produces sebum in an amount of 100 μg/cm² and above (e.g., 125, 135, 150, 175, 200 μg/cm² or higher).

In particular, 4 mg of dexmecamylamine hydrochloride is administered orally twice a day to a subject with hidradenitis suppurativa. Efficacy is measured by a change in pain, as measured using a 10-point numerical rating scale, 1 through 10, with 10 being the worst, from baseline to week 12; a change in quality of life using Dermatology Life Quality Index (DLQI) from baseline to week 12; a change in clinical status using the Hurley Staging from baseline to week 12; a change in clinical status using the International Hidradenitis Suppurative Severity Score System (IHS4) from baseline to week 12; a change in clinical status using the Hidradenitis Suppurative Clinical Response (HiSCR) from baseline to week 12; a change in clinical status using the Hidradenitis Suppurative Physician Global Assessment (HS-PGA) from baseline to week 12; a change in clinical status using the Hidradenitis Suppurative Area and Severity Index-Revised (HASI-R) from baseline to week 12; and/or a change in the absolute number of abscesses and inflammatory nodules.

### Example 5. Treatment of Seborrhea

The compound of the disclosure (e.g., dexmecamylamine substantially free of exo-R-mecamylamine) is administered to patients with seborrhea. Patients receiving the compound exhibit improved symptoms, where the seborrhea is reduced or ceased as a result of administration of the compound. In some embodiments, the subject produces sebum in an amount of 100 μg/cm² and above (e.g., 125, 135, 150, 175, 200 μg/cm² or higher).

### Example 6. Treatment of Sebaceous Hyperplasia

The compound of the disclosure (e.g., dexmecamylamine substantially free of exo-R-mecamylamine) is administered to patients with sebaceous hyperplasia. Patients receiving the compound exhibit improved symptoms, where the sebaceous hyperplasia is reduced or ceased as a result of administration of the compound. In some embodiments, the subject produces sebum in an amount of 100 μg/cm² and above (e.g., 125, 135, 150, 175, 200 μg/cm² or higher).

### Example 7. Treatment of Seborrheic Dermatitis

The compound of the disclosure (e.g., dexmecamylamine substantially free of exo-R-mecamylamine) is administered to patients with seborrheic dermatitis. Patients receiving the compound exhibit improved symptoms, where the seborrheic dermatitis is reduced or ceased as a result of administration of the compound. In some embodiments, the subject produces sebum in an amount of 100 μg/cm² and above (e.g., 125, 135, 150, 175, 200 μg/cm² or higher).

### Example 8. Treatment of Sebaceous Adenoma

The compound of the disclosure (e.g., dexmecamylamine substantially free of exo-R-mecamylamine) is administered to patients with sebaceous adenoma. Patients receiving the compound exhibit improved symptoms, where the sebaceous adenoma is reduced or ceased as a result of administration of the compound. In some embodiments, the subject produces sebum in an amount of 100 μg/cm² and above (e.g., 125, 135, 150, 175, 200 μg/cm² or higher).

### Example 9. Treatment of Sebaceous Carcinoma

The compound of the disclosure (e.g., dexmecamylamine substantially free of exo-R-mecamylamine) is administered to patients with sebaceous carcinoma. Patients receiving the compound exhibit improved symptoms, where the sebaceous carcinoma is reduced or ceased as a result of administration of the compound. In some embodiments, the subject produces sebum in an amount of 100 μg/cm² and above (e.g., 125, 135, 150, 175, 200 μg/cm² or higher).

### Example 10. Treatment of Sebaceous Cyst

The compound of the disclosure (e.g., dexmecamylamine substantially free of exo-R-mecamylamine) is administered to patients with sebaceous cyst. Patients receiving the compound exhibit improved symptoms, where a sebaceous cyst is reduced or ceased as a result of administration of the compound. In some embodiments, the subject produces sebum in an amount of 100 μg/cm² and above (e.g., 125, 135, 150, 175, 200 μg/cm² or higher).

### Example 10. Treatment of Sebaceous Wrinkles

The compound of the disclosure (e.g., dexmecamylamine substantially free of exo-R-mecamylamine) is administered to patients with wrinkles. Patients receiving the compound exhibit improved symptoms, where wrinkles are reduced or ceased as a result of administration of the compound. In some embodiments, the subject produces sebum in an amount of 100 μg/cm² and above (e.g., 125, 135, 150, 175, 200 μg/cm² or higher).

### Example 11. Treatment of Oily Skin

The compound of the disclosure (e.g., dexmecamylamine substantially free of exo-R-mecamylamine) is administered to patients with oily skin. Patients receiving the compound exhibit improved symptoms, where oil production by the skin is reduced or ceased as a result of administration of the compound. In some embodiments, the subject produces sebum in an amount of 100 μg/cm² and above (e.g., 125, 135, 150, 175, 200 μg/cm² or higher).

### Other Embodiments

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention. Other embodiments are in the claims.

What is claimed is:

1. A method of treating acne, enlarged pores, hidradenitis suppurativa, seborrhea, sebaceous hyperplasia, seborrheic dermatitis, sebaceous adenoma, sebaceous carcinoma, sebaceous cyst, wrinkles, or oily skin in a subject in need thereof, the method comprising administering to the subject a therapeutically effective amount of dexmecamylamine substantially free of exo-R-mecamylamine or a pharmaceutically acceptable salt thereof.

2. The method of claim 1, wherein the subject has acne

3. The method of claim 1, wherein the subject has enlarged pores.

4. The method of claim 1, wherein the subject has hidradenitis suppurativa.

5. The method of claim 1, wherein the subject has seborrhea.

6. The method of claim 1, wherein the subject has a sebaceous hyperplasia.

7. The method of claim 1, wherein the subject has seborrheic dermatitis.

8. The method of claim 1, wherein the subject has a sebaceous adenoma.

9. The method of claim 1, wherein the subject has a sebaceous carcinoma.

10. The method of claim 1, wherein the subject has a sebaceous cyst.

11. The method of claim 1, wherein the subject has wrinkles.

12. The method of claim 1, wherein the subject has oily skin.

13. The method of any one of claims 1-12, wherein the dexmecamylamine, or pharmaceutically acceptable salt thereof, is administered is administered by way of oral, inhalational, intranasal, or topical administration.

14. The method of any one of claim 13, wherein the subject is administered 0.1 mg to 16 mg of the dexmecamylamine substantially free of exo-R-mecamylamine, or pharmaceutically acceptable salt thereof.

15. The method of any one of claims 1-14, wherein the subject is administered 0.1 mg to 16 mg of the dexmecamylamine substantially free of exo-R-mecamylamine, or pharmaceutically acceptable salt thereof for two weeks or more.

16. The method of any one of claims 1-15, wherein dexmecamylamine hydrochloride is administered.

17. The method of claim 15 or 16, wherein the subject is administered 2 mg of the dexmecamylamine substantially free of exo-R-mecamylamine, or pharmaceutically acceptable salt thereof.

18. The method of claim 15 or 16, wherein the subject is administered 4 mg of the dexmecamylamine substantially free of exo-R-mecamylamine, or pharmaceutically acceptable salt thereof.

19. The method of any one of claims 1-18, wherein, prior to treatment, the subject produces sebum in an amount of 100 μg/cm² or greater.

20. The method of claim 19, wherein the subject produces sebum in an amount of 125 μg/cm², 135 μg/cm², 150 μg/cm², 175 μg/cm², 200 μg/cm² or greater.

21. The method of any one of claims 1-20, wherein following the treatment with dexmecamylamine substantially free of exo-R-mecamylamine, or pharmaceutically acceptable salt thereof, the subject exhibits sebum production that is at least 20% less than the sebum production in the subject prior to the treatment with the dexmecamylamine substantially free of exo-R-mecamylamine, or pharmaceutically acceptable salt thereof.

22. The method of claim 21, wherein the sebum production is facial sebum production.
